(19) United States
(12) Patent Application Publication
(10) Pub. No.: US 2015/0366952 A1
(43) Pub. Date: Dec. 24, 2015

(54) METHODS FOR REDUCING FIBROSIS INDUCED BY PERITONEAL DIALYSIS

(71) Applicant: Saint Louis University, St. Louis, MO (US)

(72) Inventors: Alireza R. Rezaie, Eureka, MO (US); Peyman Dinarvand, St. Louis, MO (US)

(21) Appl. No.: 14/747,684

(22) Filed: Jun. 23, 2015

Related U.S. Application Data
(60) Provisional application No. 62/016,482, filed on Jun. 24, 2014.

(51) Int. Cl. A61K 38/48; A61K 9/00

(52) U.S. Cl. CPC A61K 38/4866; A61K 9/0019; A61M 1/28

(57) ABSTRACT

The disclosure relates to a method of preventing, inhibiting, or reducing fibrosis, the incidence of fibrosis or the progression of fibrosis associated with peritoneal dialysis, during or after peritoneal is administered. More specifically, the methods relates to using intraperitoneal administration of activated protein C (APC) possessing cytoprotective or anti-inflammatory activity, to reduce the incidence or progression of fibrosis associated with peritoneal dialysis. The method is demonstrated using wild type APC and a mutant APC possessing cytoprotective or anti-inflammatory activity but lacking anti-coagulant activity.

### CROSS REFERENCE TO RELATED APPLICATIONS

This application claims priority to provisional application 62/016,482, filed Jun. 24, 2014, which is hereby incorporated by reference in its entirety.

### GOVERNMENT FUNDING

This work was supported by National Institutes of Health grant no. HL 101917. The government of the United States has certain rights in this invention.

### FIELD OF THE INVENTION

The invention relates to methods, for reducing the incidence of fibrosis, associated with peritoneal dialysis.

### BACKGROUND

Patients with chronic kidney disease require dialysis (hemodialysis or peritoneal dialysis) for treatment. Peritoneal dialysis is an alternative method to hemodialysis for treating patients with end-stage kidney disease. The therapeutic principle behind peritoneal dialysis is based on the use of the peritoneum as a permeable membrane where ultra-filtration and diffusion between dialysis fluid (dialysate) and toxic material, accumulated in blood, can take place across the peritoneum. However, during continuous ambulatory peritoneal dialysis, the hyperosmotic and acidic nature of dialysate causes injury to mesothelial cells of the peritoneum, thereby causing tissue fibrosis and eventually failure of the filtration. This problem affects up to 20% of patients undergoing continuous ambulatory peritoneal dialysis. The exact pathogenic mechanism of this disease process has not been elucidated and no effective therapy is available. Clinical, translational and cell-based data have indicated that long term peritoneal dialysis is associated with increased release of inflammatory cytokines including the fibrosis factor, transforming growth factor beta (TGF-β).

APC is a natural plasma anticoagulant protease that is involved in regulation of thrombin generation at vascular injury sites. APC exhibits anti-inflammatory activities when it binds to endothelial protein C receptor (EPCR) to activate a vascular cell surface receptor termed protease-activated receptor 1. APC also possesses anticoagulant activity. The Inventors have previously produced a variant of APC, using genetic engineering, which possesses APC's cytoprotective or anti-inflammatory activity, but lacks anticoagulant activity. This variant has been designated APC-2Cys.

There has been no satisfactory method for treatment of fibrosis, associated with peritoneal dialysis. The Inventors have developed a mouse model of chlorhexidine gluconate (CG)-induced peritoneal fibrosis and have used it to demonstrate how APC or a variant of APC with cytoprotective or anti-inflammatory activity (i.e., APC-2Cys), may be used to reduce fibrosis or reduce incidence of fibrosis, associated with peritoneal dialysis.

### SUMMARY OF THE INVENTION

A method of reducing the incidence of fibrosis, associated with peritoneal dialysis by injecting a subject intraperitoneally with an effective amount of an activated protein C (APC), having cytoprotective or anti-inflammatory activity, within a time reasonably close to when peritoneal dialysis is to be administered.

A method of reducing the incidence of fibrosis, associated with peritoneal dialysis by injecting a subject intraperitoneally with an effective amount of a wild type APC activity, within a time, reasonably close to when peritoneal dialysis is to be administered.

A method of reducing the incidence of fibrosis, associated with peritoneal dialysis by injecting a subject intraperitoneally with an effective amount of a mutant APC possessing cytoprotective or anti-inflammatory activity, within a time, reasonably close to when peritoneal dialysis is to be administered.

A method of reducing the incidence of fibrosis, associated with peritoneal dialysis by injecting a subject intraperitoneally with an effective amount of a mutant APC having cytoprotective or anti-inflammatory activity, but possessing reduced or lacking anticoagulant activity, within a time reasonably close to when peritoneal dialysis is to be administered.

### REFERENCE TO COLOR FIGURES

The application file contains at least one figure executed in color. Copies of this patent application publication with color photographs will be provided by the Office upon request and payment of the necessary fee.

### DESCRIPTION OF THE FIGURES

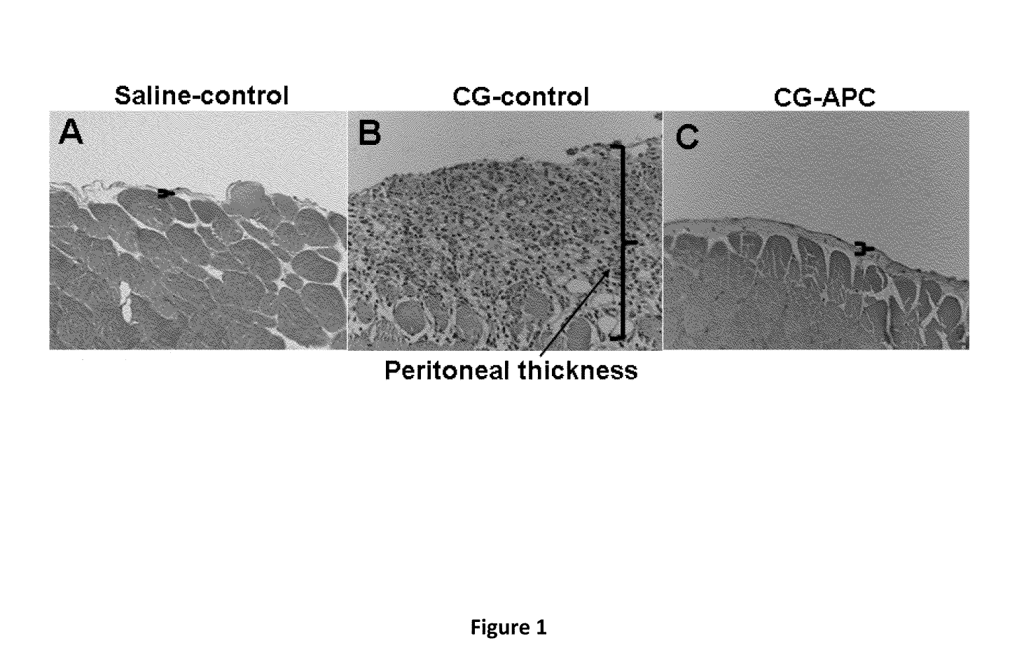

FIG. 1 illustrates histological features of peritoneal fibrosis samples by hematoxylin-eosin (H&E) staining. The saline group of mice (panel A), CG-treated group (panel B) and the APC-treated group following peritoneal fibrosis induction by CG (panel C). The arrows show the thickness of peritoneum in the CG-treated group±APC treatment.

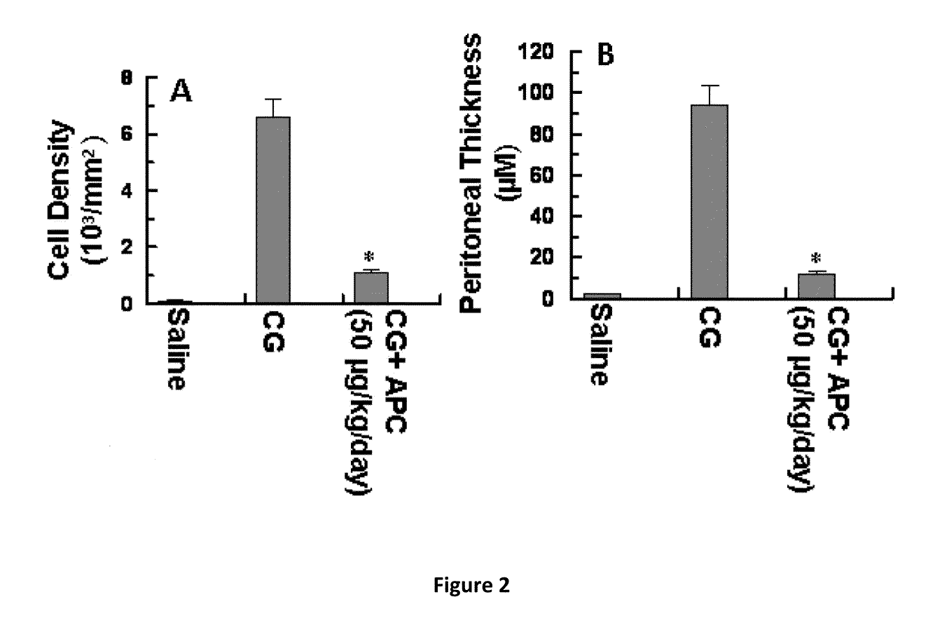

FIG. 2 illustrates the number of cells (cell density) and peritoneal thickness (μm) for saline, the CG-treated group and APC treatment are shown by bar graphs in panel A and B, respectively.

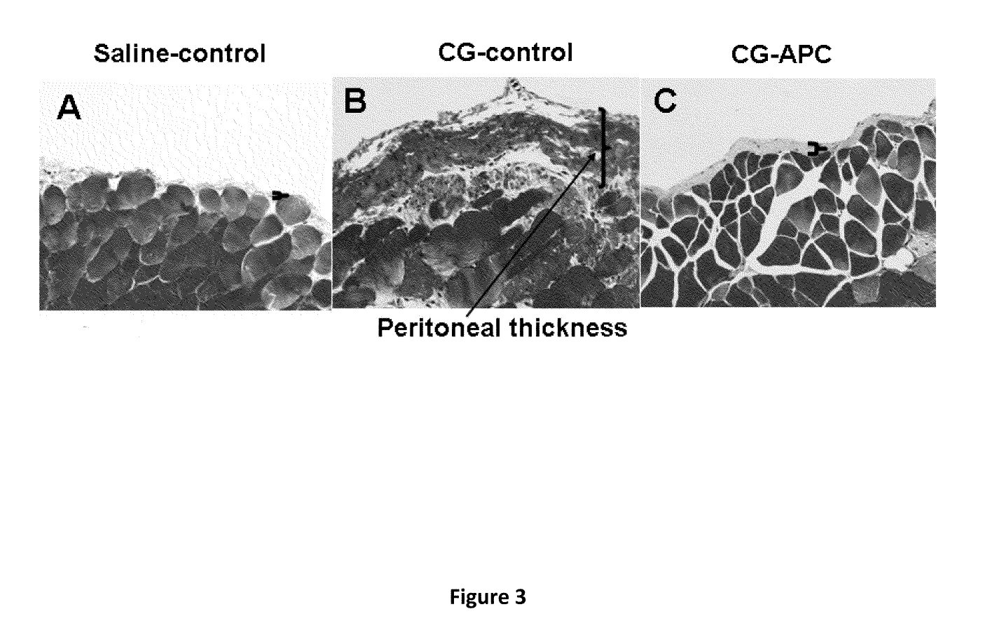

FIG. 3 illustrates histological analysis of peritoneal fibrosis samples by Masson's trichrome staining. The saline group (panel A), CG-treated control group (panel B) and the APC treated group (panel C) following CG induced peritoneal fibrosis. The arrows show the thickness of peritoneum in the saline, CG-treated group±APC treatment. The blue color represents the extracellular matrix proteins, collagens, accumulated in the peritoneal tissues of the CG-treated control which has been effectively inhibited by the APC treatment.

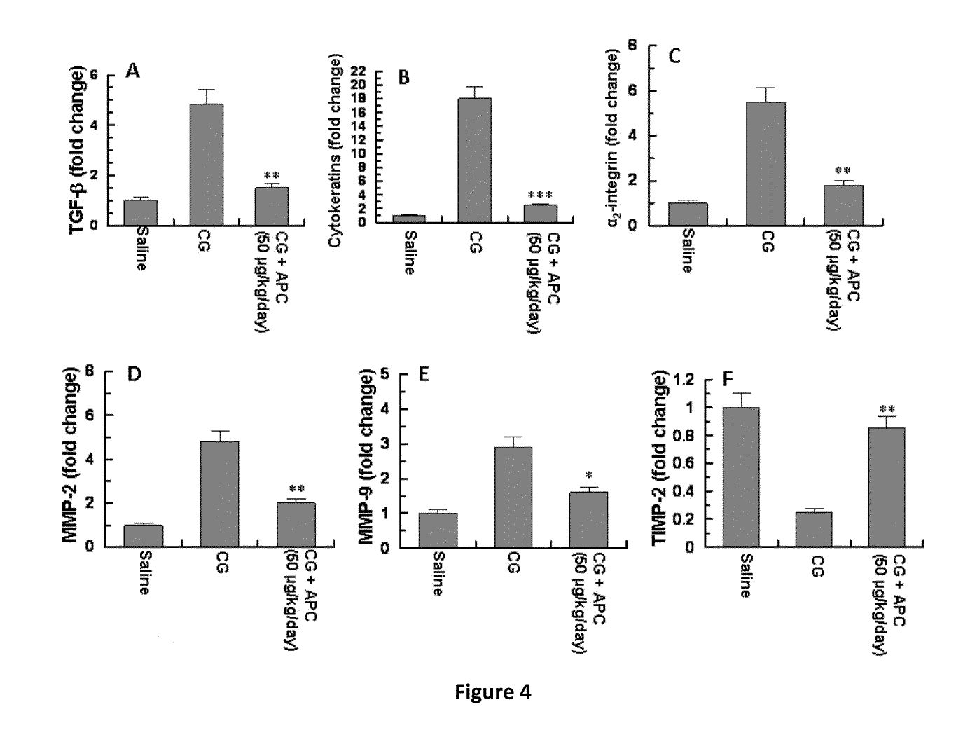

FIG. 4 illustrates analysis of mRNA expression of TGF-β, cytokratins, integrin2, MMP2, MMP9 and TIMP2 in peritoneal tissues of experimental animals. The fold enhancement in the mRNA expression levels of different molecules in control, CG-treated and CG+APC treated groups shown in panel A for TGF-β, in panel B for cytokratins, in panel C for integrin2, in panel D for MMP2, in panel E for MMP9, and in panel F for TIMP2.

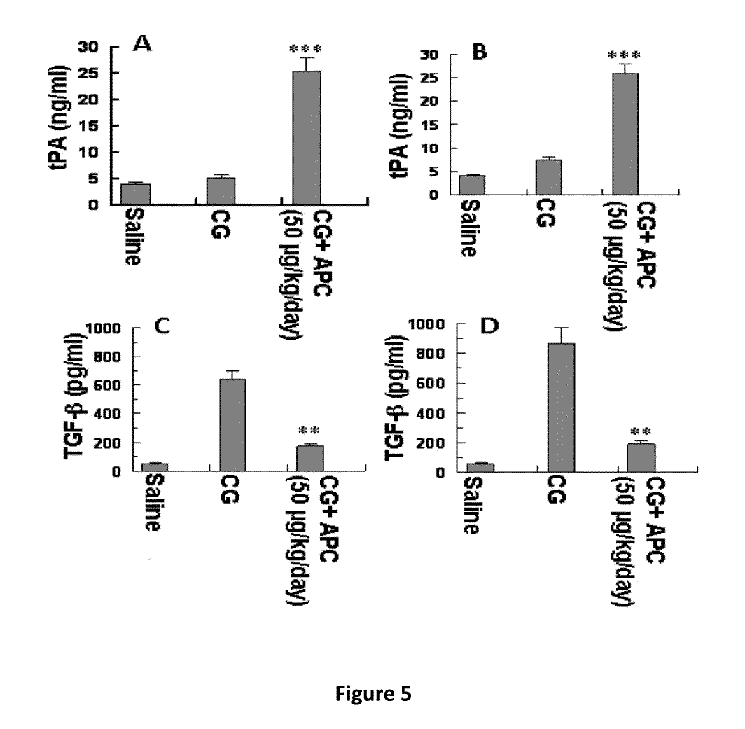

FIG. 5 illustrates Analysis of protein expression level of tPA and TGF-β in peritoneal tissues of animals by ELISA assays. The expression of tPA is dramatically enhanced in the APC treated group in both day 10 (panel A) and day 21 (panel B) of the CG-treated animals. By contrast the expression of TGF-β is dramatically reduced in the APC treated group in both day 10 (panel A) and day 21 (panel B) of the CG-treated animals. These mediators have been evaluated in peritoneal fluid of mice.

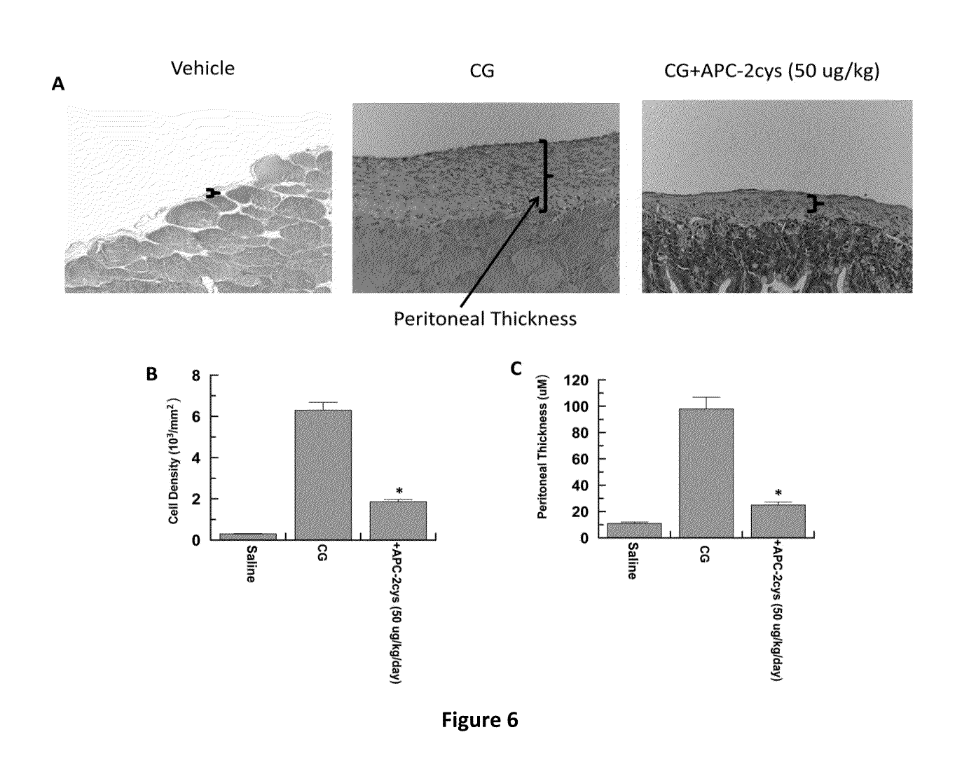

FIG. 6 illustrates the effect of APC-2Cys on CG induced peritoneal fibrosis. Hematoxylin-eosin staining revealed that the mice, which were injected with only CG, exhibit a dramatic an increase in thickening of the peritoneum by day 21 compared to the vehicle group (panel A). By contrast, in the APC-2Cys-treated group, the CG-induced thickening of the peritoneum was dramatically inhibited by day 21 compared to CG group (panel A). Quantitative analysis of these results (panels B and C) clearly suggests that APC-2Cys is a potent inhibitor of CG-induced peritoneal fibrosis.

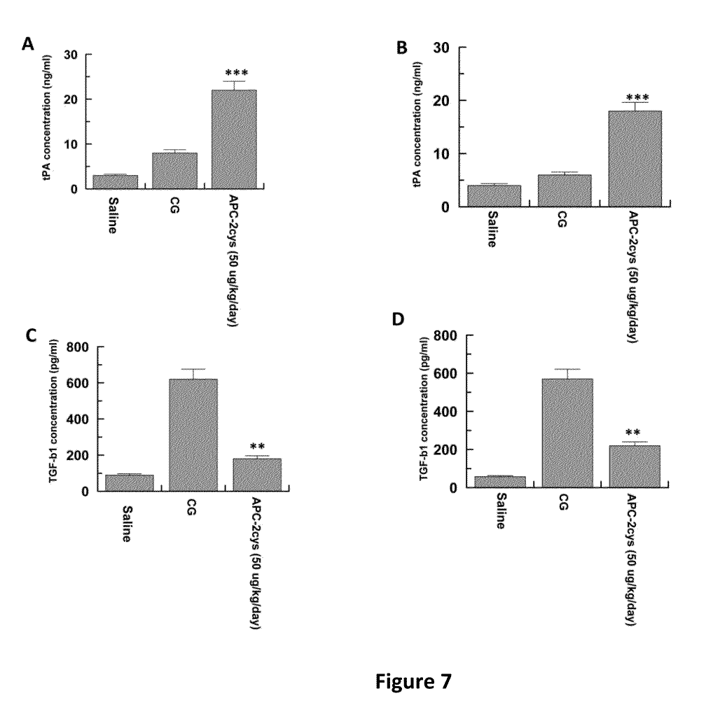

FIG. 7 illustrates the effect of APC-2Cys on levels of the anti-fibrosis proteinase, tissue-type plasminogen activator (tPA), measured on both day-10 (panel A) and day-21 (panel B) in CG (only) and APC-2Cys (APC-2Cys+CG)-treated mice. Enzyme-linked immunosorbent assay indicated that tPA was elevated in the APC-2Cys group compared CG controls. This is constant with APC-2Cys reducing fibrosis in these animals. FIG. 6 also illustrated the effects of APC-2Cys on levels of TGF-β in CG (only) and APC-2Cys (APC-2Cys+CG) treated mice at both day-10 (panel C) and day-21 (panel D). The results indicate that TGF-β is reduced in APC-2Cys treated mice. This is constant with APC-2Cys reducing fibrosis in these animals. These mediators have been evaluated in peritoneal fluid of mice.

### DETAILED DESCRIPTION OF THE INVENTION

The invention provides a novel approach for preventing, inhibiting, or reducing fibrosis, the progression of fibrosis, or the incidence of fibrosis, associated with peritoneal dialysis that may form during or after peritoneal dialysis takes place.

Peritoneal dialysis (PD) is a treatment for patients with severe chronic kidney disease. It may be used as an alternative to hemodialysis. Dialysate is infused into the peritoneal cavity where electrolytes, urea, glucose, albumin and other small molecules, are exchanged from the blood across the peritoneum. The dialysate is removed and replaced to repeat the process. A non-limiting example of the procedure includes Continuous Ambulatory Peritoneal Dialysis (CAPD) which may be administered at a patient's home, and requires an average of 4-5 dialysate exchanges each day, each exchange requiring about 30-45 minutes. Another non-limiting example includes Continuous Cycling Peritoneal Dialysis (CCPD) which uses an apparatus to mediate a continuous exchange of dialysate which takes about 9-10 hours, and is administered during the day or overnight. Occasionally some of these patients will require an additional exchange during the day or evening as well. Peritoneal dialysis procedures typical employ a flexible tube or port, which is inserted through the patient's abdomen wall to allow access to the peritoneum and peritoneal cavity. This port may also be used to administer APC to prevent fibrosis associated with peritoneal dialysis.

Without wishing to be bound by theory, it is believed that peritoneal fibrosis begins during or after dialysis due to trauma associated with the dialysate being in continuous contact with the peritoneum. It is thought that trauma caused by the dialysate results in denudation of mesothelial cells, tissue fibrosis, hyalinizing vasculopathy, and eventual failure of filtration. It also results in an increase in pro-inflammatory cytokines and the infiltration of inflammatory cells including pathologic fibroblasts, and myofibroblasts. The Inventors reasoned that if this inflammatory cascade of events can be reduced or prevented following PD, then subsequent fibrosis may be reduced or eliminated.

Activated protein C (APC) is a natural anticoagulant and anti-inflammatory serine protease found in plasma. Activated protein C is well known for its cytoprotective and anticoagulative properties. The Inventors have previously disclosed the use of wild type APC to reduce the incidence of post-surgical adhesions (see U.S. application Ser. No. 13/854,117, incorporated herein by reference in its entirety). Since that disclosure, using genetically engineered variants of APC that were selectively deficient in functional properties, the Inventors made the surprising discovery that a variant of APC, designated APC-2Cys, which retained its cytoprotective activity but lacked anticoagulant activity, was as effective as wild-type APC in reducing post-surgical adhesions. The Inventors further confirmed this discovery by testing other genetically engineered APC variants, and found that variants which lacked cytoprotective activity had poor anti-adhesive activity.

The Inventors reasoned that APC or variants of APC with cytoprotective activity, including APC 2Cys, may be effective in reducing fibrosis, if injected into the peritoneal, before, during, or within a time period reasonably close to when peritoneal dialysis is to be administered. By way of example, many human subjects undergoing peritoneal dialysis receive this treatment daily. An effective dose of APC, or a variant of APC with cytoprotective activity, by way of example APC-2Cys, may also be administered daily, or on each day the subject receives PD over the course of treatment. Preferably, an aqueous solution containing an effective amount of APC is injected at a time reasonably close to when peritoneal dialysis is administered. In a preferred embodiment, a time reasonably close to when PD is administered, may be about 15 minutes, about 30 minutes, about 45 minutes, about 1 hour, about 2 hours or about 4 hours before or after peritoneal dialysis is administered. More preferably, an effective amount of APC may be injected into the peritoneal cavity, concurrent with peritoneal dialysis, or about 30 minutes, about 45 minutes, about 1 hour, or about 2 hours before peritoneal dialysis is administered. Most preferably an effective amount of APC may be injected into the peritoneal cavity about 1 hour before peritoneal dialysis is administered.

Using an animal model of peritoneal fibrosis, the Inventors have demonstrated that both wild type APC and APC-2Cys are effective at reducing the incidence of fibrosis, associated with peritoneal dialysis. Since the biological activity that is common to both wild type APC and APC 2Cys in preventing peritoneal fibrosis is its cytoprotective or anti-inflammatory activity. It is anticipated other variants of APC that retain APC cytoprotective or anti-inflammatory activity will be effective in reducing the incidence of fibrosis, associated with peritoneal dialysis.

The Inventors used a model of peritoneal trauma where trauma was induced chemically in the peritoneal cavity of mice by intraperitoneal injection of chlorhexidine gluconate (CG). This was performed with or without a subsequent intraperitoneal injection APC or APC variant. Using this model the Inventors demonstrated that APC injected intraperitoneally resulted in a reduction in peritoneal fibrosis. A reduction in fibrosis was evident by both histology, as well as accumulation of collagen and extracellular matrix proteins. Reduction in peritoneal fibrosis was also indicated by a reduction in the inflammatory indicators known to accompany peritoneal fibrosis.

Therefore, one embodiment of the invention is a method of reducing fibrosis or the incidence of fibrosis, associated with peritoneal dialysis by injecting intraperitoneally, an effective amount of activated protein C, at a time reasonably close to when, peritoneal dialysis is administered.

In another embodiment, the invention is a method of reducing the fibrosis or the incidence of fibrosis, associated with peritoneal dialysis by injecting intraperitoneally, an effective amount of activated protein C, or a variant of activated protein C with cytoprotective or anti-inflammatory activity, by way of example APC-2Cys, within a time reasonably close to when peritoneal dialysis is administered.

In yet another embodiment, of the invention is a method of reducing the fibrosis or the incidence of fibrosis, associated with peritoneal dialysis by injecting intraperitoneally, an effective amount of a variant of activated protein C with cytoprotective or anti-inflammatory activity, but with reduced or lacking anticoagulant activity, by way of example APC-2Cys, within a time reasonably close to when peritoneal dialysis is administered.

### I. Activated Protein C and Activated Protein C-2Cys.

Protein C (PC) and Activated Protein C (APC)

Wild type human protein C is the inactive zymogen form of a vitamin K-dependent plasma serine protease. Protein C, in vivo, or as secreted by a eukaryotic cell in culture, exists in the form of a disulfide-linked two chain molecule. It is transcribed as a single polypeptide, (see SEQ ID NO:1), which then undergoes post-transitional modification. Modifications include removal of a signal peptide sequence (amino acids 1-42), and removal of a dipeptide sequence (amino acids 198-199), which produces two polypeptides referred to as the light (˜25 kD) (amino acids 43-197) and heavy chains (˜41 kD)(amino acids 200-461). Variations in molecular weight occur due to differences in glycosylation, which is also a post-translational modification. The light chain contains a region of gamma-carboxyglutamic acid, which is required for membrane binding and is dependent on Ca²⁺. The heavy chain contains the serine protease domain, which also contains a Ca²⁺ binding site described in detail below. The heavy chain also contains the activation peptide. Activation of protein C to activated protein C takes place in vivo by removal of this activation peptide (amino acids 200-211) by thrombin. A disulfide bond at cysteine 183 and cysteine 319 connects the heavy and light chains. (Plutzky et al., (1986) Proc. Natl. Acad. Sci. (USA) 83, 546-550)

i) Activation and Anticoagulation Activity

In vivo, protein C circulates as an inactive zymogen. Activation of protein C to activated protein C takes place by proteolytic removal of the activation peptide (amino acids 200-211, see SEQ ID NO:1), from the heavy chain. Protein C is activated on the surface of endothelial cells by a thrombin-thrombomodulin (thrombin-TM) complex, which is also accelerated by the endothelial protein C receptor (EPCR). This is believed to take place by co-localizing protein C with the thrombin-TM complex on the endothelial cell surface (Stearns-Kurosawa et al. (1996) Proc Natl Acad Sci. (USA); 93:10212-10216). After activation, activated protein C down-regulates the clotting cascade via a feedback loop mechanism (Stenflo J. (1984) Thromb Hemost. 10:109-121; Esmon C T. (1993) Thromb. Haemost. 70:1-5). Once protein C is activated it may dissociate from EPCR, and form a complex with the vitamin K-dependent protein cofactor, protein S. This complex will shut down the generation of thrombin derived from the cofactor effect of factors Va (Na) and VIIIa, which are known to be procoagulant cofactors of the prothrombinase and intrinsic Xase complexes, respectively.

i) Cytoprotective, Anti-Inflammatory Properties, and Anti-Apoptotic Activity

In addition to providing anti-coagulant activity, APC possesses cytoprotective, anti-inflammatory, and anti-apoptotic proprieties. The term “cytoprotective” as used herein is meant to refer collectively to these protective properties or activities, meaning the cytoprotective, anti-inflammatory, and anti-apoptotic proprieties or activities of APC and variants of APC such as APC 2Cys. When APC is associated with EPCR, it elicits protective signaling responses in endothelial cells (Taylor et al. (1987) J Clin Invest. 1987; 79:918-925; Taylor et al. (2000) Blood; 95:1680-1686; Joyce et al. (2001) J Biol Chem.; 276:11199-11203; Ruf et al. (2003) J Thromb Haemost. 1:1495-1503; Mosnier et al. (2004) Blood. 104:1740-1744; Finigan et al. (2005) J Biol Chem.; 280:17286-17293). These protective signals may account for the beneficial effects associated with APC when used as an anti-inflammatory agent for treating severe sepsis patients (Bernard et al. (2001) N Eng J Med.; 344:699-709). The mechanisms of the anti-inflammatory and cytoprotective effects of APC are not well understood, however, it is believed that an APC/EPCR complex cleaves protease-activated receptor-1 (PAR-1) to initiate protective signaling events in endothelial cells (Ruf et al. (2003) J Thromb Haemost. 1:1495-1503; Mosnier et al. (2004) Blood. 104:1740-1744). PAR-1 cleavage by APC may also be required for the inhibition of apoptosis in human brain endothelial cells induced by hypoxia (Cheng et al. (2003) Nature Med.; 9:338-342).

ii) Anticoagulant Activity

The mechanism through which protein C, once activated, functions in the anti-coagulant pathway has been extensively studied and is well understood (Walker et al. (1992) FASEB J; 6:2561-2567). After activation, APC may dissociate, from EPCR and bind to protein S, where it functions as an anticoagulant by degrading factors Va and VIIIa. Specific recognition of procoagulant factors Va and VIIIa, is determined by the basic residues of an APC exosite (Friedrich et al. (2001) J Biol Chem.; 276:23105-23108; Manithody et al. (2003) 101:4802-4807; Gale et al. (2002) J Biol Chem.; 277:28836-28840). These basic residues are clustered on three exposed surface loops referred to as 37-39 loop, 60-68 loop and 70-80 loop (chymotrypsin numbering system) (Bode et al. (1989) EMBO J.; 8:3467-3475). These basic residues constitute a binding site for TM in the thrombin-TM complex. With the exception of the 60 loop, they are also involved in recognition and subsequent degradation of factors Va and VIIIa by APC in the anti-coagulant pathway (Friedrich et al. (2001) J Biol Chem.; 276:23105-23108; Manithody et al. (2003) Blood; 101:4802-4807; Gale et al. (2002) J Biol Chem.; 277:28836-28840).

### Protein C-2Cys and Activated Protein C-2Cys

The instant invention utilizes a variant of the wild type protein C as fully described in U.S. Pat. No. 7,785,857, also referred to therein as cross-linked protein C and cross-linked activated protein C. U.S. Pat. No. 7,785,857 is incorporated herein by relevance in its entirety. The instant invention refers to this variant as protein C-2Cys (PC-2Cys) and activated protein C-2Cys (APC-2Cys) respectively. Protein C-2Cys may be activated by thrombin to form activated protein C-2Cys. Activated protein C-2Cys demonstrates the cytoprotective properties of wild-type APC without the anticoagulant properties.

Protein C-2Cys is produced by engineering a disulfide bond to form a cross-link between two anti-parallel β-sheets of the heavy chain polypeptide of wild-type human protein C. Specifically, amino acid residues at position 264 and also position 279 of wild type protein C (SEQ ID NO:1) are each substituted with a cysteine, to produce a novel polypeptide (SEQ ID NO:2) (Bae et al. (2007) J Biol Chem.; 282: No. 12: 9251-9259). The presence of cysteines in these positions will allow the formation of an intra-chain disulfide bond post-translationally, thereby forming a cross-link between these two amino acids within the anti-parallel β-sheets of residues 261-266 and 278-288 of the protein C heavy chain. Similarly, the presence of cystines or another cross-linking agent, installed at one or more of these positions, within or near these anti-parallel β-sheets would produce a similar result. Between these anti-parallel β-sheets is the Ca²⁺ binding 70-80 loop (CHT). While not agreeing to be bound by theory, one hypothesis is that the binding of Ca²⁺ to the 70-80 loop of protein C is associated with a conformational change in the zymogen that is optimal for interaction with thrombin in the presence of TM but inhibitory for interaction in the absence of the cofactor (Yang et al. (2006) Proc Natl Acad Sci. (USA); 103:879-884). By cross-linking these two anti-parallel β-sheets, the 70-80 calcium binding loop becomes stabilized and no longer binds Ca²⁺. The engineered disulfide bond also stabilizes a Na+ binding site in the high affinity state. These Ca²⁺ and Na⁺ binding sites modulate activity of protein C and may be necessary for the amidolytic activity and proteolytic activity demonstrated by APC, as described in detail below. The light chain of Protein C-2Cys is not modified, and as in wild type, remains bound to the heavy chain by a single disulfide bond.

Therefore, by specifically engineering specific regions of the protein C polypeptide, while leaving other regions intact, the anti-coagulant activity of Activated Protein C-2Cys is essentially abolished, while it's anti-inflammatory and cytoprotective signaling properties remain intact.

i) Activation of Protein C-2Cys

Wild type protein C and protein C-2Cys are activated by free thrombin to form activated Protein C and activated protein C-2Cys respectively. Wild type protein C is activated by proteolytic removal of the activation peptide, amino acids 200-211 (SEQ ID NO:1) of the heavy chain. The activation peptide is not altered in protein C-2Cys and activation takes place by thrombin cleavage or removal of the same activation peptide.

ii) Cytoprotective Properties of Activated Protein C-2Cys

As described above, wild type APC possesses protective properties including cytoprotective, anti-inflammatory, and anti-apoptotic activity, collectively referred to herein as cytoprotective properties or cytoprotective activities. These activities are mediated through a receptor designated endothelial protein C receptor (EPCR) present on endothelial cells as well as other cell types. EPCR binding which takes place via the Gla domain (amino acids 43-88 of SEQ ID NO:1) The Gla domain is not altered in Protein C-2Cys, and biological activity of the Gla domain remains unchanged. Therefore, similar to wild type, EPCR binding to endothelial or other cell types via the Gla domain still occurs. Activated Protein C-2Cys participates in cytoprotective, cell signaling, anti-inflammatory and anti-apoptotic activities in the same manner as wild-type APC. These properties are further described and demonstrated in U.S. Pat. No. 7,785,857.

### Method of Making PC-2Cys and APC-2Cys

The method for making PC-2Cys involves methodology that are generally well known and described in detail in numerous laboratory protocols, one of which is Molecular Cloning 3rd edition, (2001) J. F. Sambrook and D. W. Russell, ed., Cold Spring Harbor University Press. Many modifications and variations of the present illustrative DNA sequences and plasmids are possible. For example, the degeneracy of the genetic code allows for the substitution of nucleotides throughout polypeptide coding regions, as well as in the translational stop signal, without alteration of the encoded polypeptide coding sequence. Such substitutable sequences can be deduced from the known amino acid or DNA sequence of human protein C and can be constructed by following conventional synthetic or site-directed mutagenesis procedures. Synthetic methods can be carried out in substantial accordance with the procedures of Itakura et. al., (1977) Science 198:1056 and Crea et. al. (1978) Proc. Natl. Acad. Sci, USA 75:5765. Therefore, the present invention is in no way limited to the DNA sequences and plasmids specifically exemplified.

i) Insertion of Cysteines into Wild Type APC or Derivations of Wild Type APC

A polynucleotide encoding human protein C polypeptide (SEQ ID NO:1) or variants thereof, may be engineered whereby the codons representing one or more of amino acids 261 to 266 and one or more of amino acids 278 to 288 are replaced with codons for cysteine. By way of non-limiting example, and as described below in the examples, nucleotide sequences complementary for a polynucleotide encoding these amino acid sequences may be constructed whereby codons representing amino acid 264 and amino acid 279 are substituted by a cysteine. One of ordinary skill in the art will understand that other codons representing other or additional amino acids within these complementary nucleotide sequences may be replaced with codons for cysteine to provide alternative or additional cysteine substitutions in a similar manner. Examples of primers described below in the examples. The polynucleotide may then be amplified using standard PCR mutagenesis methods as previously described (Rezaie et al. (1992) Journal of Biological Chemistry, vol. 267, pp. 26104-26109) and herein incorporated by reference. The resulting mutant or variant of protein C cDNA may be sub-cloned and inserted into a suitable expressions vector using a number of commercially available restrictions enzymes and expressed in a wide variety of eukaryotic, especially mammalian, host cells. The polynucleotide may be operable linked to a number of suitable control elements to provide an expressible nucleic acid molecule by using standard cloning or molecular biology techniques. See, e.g., Edge (1981) Nature 292:756; Nambair et al. (1984) Science 223:1299; and Jay et al. (1984) J. Biol. Chem. 259:6311. Examples of expression vectors that may be effective for the expression of Protein C-2Cys include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSHIPERV (Stratagene, La Jolla Calif.), and PTET-OFF, PTET-ON, PTRE2, PTRE2-LUC, PTK-HYG (Clontech, Palo Alto Calif.). Protein C-2Cys may be expressed using (i) a constitutively active promoter, (e.g., from cytomegalovirus (CMV), Rous sarcoma virus (RSV), SV40 virus, thymidine kinase (TK), or P.beta.actin genes), (ii) an inducible promoter (e.g., the tetracycline-regulated promoter (Gossen et al. (1992) Proc. Natl. Acad. Sci. USA 89:5547-5551; Gossen, M. et al. (1995) Science 268:1766-1769; Rossi et al. (1998) Curr. Opin. Biotechnol. 9:451-456), commercially available in the T-REX plasmid (Invitrogen).

Once constructed, the expression vector encoding Protein C-2Cys may be transfected into host cells using standard gene delivery protocols. Methods for gene delivery are known in the art, and include but are not limited to methods based on naked nucleic acids, calcium phosphate, electroporation, microinjection liposomes, cells, retrovirus including lentiviruses, adenovirus and parvoviruses including adeno-associated virus herpes simplex virus. See, e.g., U.S. Pat. Nos. 7,173,116 6,936,272, 6,818,209, and 7,232,899, which are hereby incorporated by reference. Other gene delivery mechanisms include liposome-derived systems, artificial viral envelopes, and other systems known in the art (See, e.g., Rossi, J. J. (1995) Br. Med. Bull. 51(1):217-225; Boado, R. J. et al. (1998) J. Pharm. Sci. 87; Mosnier et al. (2004) Blood. 104:1740-1744):1308-1315; and Morris, M. C. et al. (1997) Nucleic Acids Res. 25(14):2730-2736).

Techniques for maintaining cells in culture to allow the expression of recombinant polypeptides are well known. By way of example the polynucleotide described above may be expressed in human embryonic kidney cells (HEK-293) using the RSV-PL4 expression system purification vector system as described (Yang et al. (2006) Proc Natl Acad Sci. (USA); 103:879-884) and Yan, U.S. Pat. No. 4,981,952, both of which are hereby incorporated by reference.

Protein C-2Cys may be harvested from the culture media and purified through any combination of protein purification techniques known in the art including various immuno-affinity techniques. An antibody directed to almost any epitope on Protein C-2Cys may be immobilized to a support structure. A physiological solution containing the molecule to be purified is exposed to the antibody whereby the target molecule is bound by the antibody. Methods of releasing polypeptides from antibodies are also well known and may include changes in pH, and elution with various salts, metal ions, EDTA, EGTA, or detergents.

Activated Protein C-2Cys may be produced from protein C-2Cys by incubation with a proteolytic enzyme such as thrombin in a physiological solution. By way of example a solution containing physiological salts and protein C-2Cys may be passed over a column comprising thrombin immobilized to Sepharose. Alternatively, activated protein C-2Cys may be produced directly by expression of a polynucleotide engineered to transcribe an activated protein C-2Cys.

ii) Variants of APC and APC-2Cys

The disclosure of APC and APC-2Cys are meant to be exemplary only. One of ordinary skill in the art will appreciate that variants of APC or APC-2Cys will be effective in reducing fibrosis or the incidence of fibrosis associated with peritoneal dialysis provided they retain cytoprotective properties or activities such as anti-inflammatory and anti-apoptotic activities. Variants of APC may be modified by the insertion of cystines in the 70-80 calcium binding loop, by way of example at positions 264 and 279 to eliminate anticoagulant activity. Variants of APC may also be produced by the insertion, deletion or substitution, of one or more amino acids at other sites in APC provided the resulting polypeptide retains cytoprotective activity. Variants of APC may also be produced by applying similar modifications to APC-2Cys, or other existing variants of APC. It is expected that these modified derivative polypeptides would possess the same or similar anti-adhesive activity as has been demonstrated for APC and PC-2Cys, provided the resulting polypeptide retains cytoprotective activity.

Examples of variants or derivatives of APC that may be useful in reducing fibrosis or the incidence of fibrosis associated with peritoneal dialysis are any variants or mutants of APC which possess cytoprotective, cell signaling, anti-inflammatory or anti-apoptotic activities including those described by Gerlitz, et al., U.S. Pat. No. 5,453,373, and Foster, et al., U.S. Pat. No. 5,516,650, and Griffin, et al., U.S. Pat. No. 7,498,305, the entire teachings of which are hereby included by reference.

Other examples of variants or mutants or derivatives of APC, or APC-2Cys include APC or APC-2Cys with conservative amino acids substitutions that retain cytoprotective properties such as anti-inflammatory and anti-apoptotic activities, regardless of the presence or absence of anticoagulant activity. These variants, mutants, or derivatives of APC, or APC-2Cys are expected to be effective in reducing fibrosis or the incidence of fibrosis associated with peritoneal dialysis when applied according to the methods described herein.

Conservative amino acid substitutions are well known in the art (see Creighton (1984) Proteins. W. H. Freeman and Company (Eds)). Examples of conservative amino acid substitution may refer to the replacement of amino acids of a polypeptide with other amino acids having similar properties, by way of example, similar hydrophobicity, hydrophilicity, or overall charge. For examples of conservative amino acid substitutions see Table 1.

TABLE 1
Examples of Conservative
amino acid substitutions.

| | Conservative |
|---|---|
| Amino Acid | Substitution |
| Ala | Ser |
| Arg | Lys |
| Asn | Gln, His |
| Asp | Glu |
| Gln | Asn |
| Cys | Ser |
| Glu | Asp |
| Gly | Pro |
| His | Asn, Gln |
| Ile | Leu, Val |
| Leu | Ile, Val |
| Lys | Arg, Gln |
| Met | Leu, Ile |
| Phe | Met, Leu, Tyr |
| Ser | Thr, Gly |
| Thr | Ser, Val |
| Trp | Tyr |
| Try | Trp, Phe |
| Cal | Ile, Leu |

### Production of APC and APC 2-Cys: Construction and Expression of Recombinant Proteins

A method of making and producing APC (wild type) and APC-2Cys was performed as described in U.S. Pat. No. 7,785,857. APC-2Cys is referred to therein as cross-linked APC. U.S. Pat. No. 7,785,857 is incorporated herein by reference in its entirety, The amino acid sequence for Protein C has previously been described (Beckmann et al. (1985) Nucleic Acids Res. vol. 13 pp. 5233-5247; Plutzky et al. (1986) PNAS Vol. 83, pp 546-550), and is represented by SEQ ID NO:1 (Accession number NP_{—}000303, NBCI). Elements of the inventors' methodology not described herein are generally well known and detailed in numerous laboratory protocols, including Molecular Cloning 2nd edition, (1989) Sambrook, J., Fritsch, E. F., and Maniatis, J., Cold Spring Harbor., and Current Protocols in Molecular Biology, volumes 1-3, John Wiley & Sons, Inc. herein incorporated by reference.

Wild-type protein C (SEQ ID NO:1) and the Protein C-2Cys, (SEQ ID NO:2) were expressed in human embryonic kidney cells (HEK-293) by using the RSV-PL4 expression system purification vector system as described (Yang et al. (2006) Proc Natl Acad Sci. (USA); 103:879-884) and herein incorporated by reference. Two complementary sense 5′-AAG AAG CTC CTT GTC TGC CTT GGA GAG TAT GAC-3′ (SEQ ID NO:3) and antisense 5′-GTC ATA CTC TCC AAG GCA GAC AAG GAG CTT CTT-3′ (SEQ ID NO:4) oligonucleotide PCR primers representing the three base codons for the amino acid residues 62-72 (chymotrypsin numbering) were synthesized in which the codon for Arg-67 (residue 264 of SEQ ID NO:1) was replaced with the codon for cysteine in both primers (underlined). Moreover, two additional oligonucleotides 5′-GAG AAG TGG GAG CTG TGC CTG GAC ATC AAG GAG-3′ (SEQ ID NO:5) (sense) and 5′-CTC CTT GAT GTC CAG GCA CAG CTC CCA CTT CTC-3′ (SEQ ID NO:6) (antisense) representing amino acid residues 77-87 were synthesized in which the codon for the residue Asp-82 (residue 279 of SEQ ID NO:1) was replaced with the codon representing cysteine in both primers (underlined) The protein C cDNA (SEQ ID NO:7) (Accession number NM_{—}000312) was amplified in two rounds to incorporate the desired mutations into the protein C sequence using standard PCR mutagenesis methods as previously described (Rezaie et al. (1992). Journal of Biological Chemistry, vol. 267, pp. 26104-26109) and herein incorporated by reference. The resulting mutant protein C cDNA (SEQ ID NO:8) was sub-cloned into HindIII and XbaI restriction enzyme cloning sites of the commercially available expression vector pRc/RSV (Invitrogen, San Diego, Calif.) using standard cloning methods. This vector contains a G418 resistant gene for selection in mammalian cells using the aminoglycoside antibiotics Gentamycin (Calbiochem, San Diego, Calif.). The accuracy of the mutations in the expression vector containing the mutant cDNA was confirmed by DNA sequencing and then introduced to human embryonic kidney (HEK) 293 cells for expression. A high expressing stable G418 resistant clone was identified by a Sandwich ELISA using an anti-protein C polyclonal antibody and the HPC4 monoclonal antibody, and expanded for production as described (Ref. Journal of Biological Chemistry, A. R. Rezaie and C. T. Esmon, vol. 267, pp. 26104-26109, 1992). The mutant protein APC-2Cys (SEQ ID NO:2) was isolated from 20-L cell culture supernatants by a combination of immunoaffinity and ion exchange chromatography using the HPC4 monoclonal antibody and a Mono Q ion exchange column (Amersham Pharmacia). The preparation of wild-type protein C and protein C-2Cys as well as APC-E170A and APC-S195A are described in Refs. Bae J S, Yang L, Manithody C, and Rezaie A R. (2007), J. Biol. Chem. 282:9251-9259 and Bae J S, Yang L, Manithody C, and Rezaie A R (2007) J. Biol. Chem. 2007, 282(35):25493-25500, incorporated herein by reference in their entirety.

### II. Formulations and Administration of Activated Protein C-2Cys (APC-2Cys)

### A. Pharmaceutical Dosage Form

Pharmaceutically acceptable formulations are well known for use as injectable solutions of APC. It is expected that most known injectable physiological solutions or formulations will be acceptable for the topical peritoneal application described herein. However, the amounts of APC per milliliter of solution may vary widely. By way of example, an effective amount of APC which may be obtained commercially as a sterile solution and diluted into a larger volume, or adequate volume, of sterile aqueous solution to be applied as described herein.

APC is a hydrophilic polypeptide and may be administered in a sterile aqueous solution, preferably in a physiological solution. A physiological solution may be comprised of isotonic balanced salts with a pH of about 7.0 to about 7.5. A preferred physiological solution may comprise isotonic saline and a pH of 7.5.

The aqueous solution may further contain various salts or buffers that are well known in the art. Injectable preparations, for example, sterile aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Non-limiting examples of acceptable vehicles and solvents that may be employed include, Ringer's solution, or isotonic sodium chloride solution.

It is preferable to maintain the pH in a physiological range, from about 6.0 to about 6.5, or about 6.5 to about 7.0, or about 7.0 to about 7.5, or about 7.5, to about 8.0, or most preferably about 7.0 to about 7.5. To maintain effective pH control, the activated protein C solution should contain a pharmaceutically acceptable buffer.

Similarly, it is preferable to maintain the ionic strength in a physiological range. The ionic strength is generally determined by the salt concentration of the solution. Pharmaceutically acceptable salts typically used to generate ionic strength include, but are not limited to, potassium chloride (KCl) and sodium chloride (NaCl). The preferred salt is maintained in a physiological range; for example, sodium chloride may be used at a concentration of 0.9 percent by weight or 100 to 150 mM.

Formulations developed for activated protein C are also known in the art and including those described in U.S. Pat. Nos. 6,630,137, 6,159,468, and 6,359,270 which are hereby incorporated by reference. Activated protein C may be formulated to prepare a pharmaceutical composition comprising as the active agent, protein C or activated protein C, and a pharmaceutically acceptable solid or carrier. For example, a desired formulation would be one comprising a bulking agent such as sucrose, a salt such as sodium chloride, a buffer such as sodium citrate and activated protein C. Formulations may be lyophilized for storage, and hydrated before use. Examples of stable lyophilized formulations include 5.0 mg/ml activated protein C, 30 mg/ml sucrose, 38 mg/ml NaCl and 7.56 mg/vial citrate, pH 6.0; and, 20 mg/vial activated protein C, 120 mg/ml sucrose, 152 mg/vial NaCl, 30.2 mg/vial citrate, pH 6.0.

Alternatively, APC formulated into pharmaceutical compositions are administered by a number of different means that will deliver a therapeutically effective dose. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

### B. Administration of Therapeutically Effective Amounts

A therapeutically effective amount, also referred to herein as an effective amount of APC, or a variant of APC with intact cytoprotective properties, by way of example APC 2Cys, may be determined by a skilled practitioner, typically a medical doctor. A therapeutically effective amount or an effective amount may be administered in an appropriate volume of aqueous solution by injection or infusion, possibly through a flexible port which may be installed through the abdominal cavity to facilitate peritoneal dialysis. An effective amount may be calculated based on the subject's body weight. By way of example, an effective amount may be between about 0.01 and about 1000 micrograms per kilogram of the subject being treated. A preferred effective amount is between about 1 and about 500 micrograms per kilogram of the subject being treated. A more preferred effective amount is between about 10 and about 200 micrograms per kilogram of the subject being treated. Another more preferred effective amount is between about 25 and about 200 micrograms per kilogram of the subject being treated. Another more preferred effective amount is between about 25 and about 100 micrograms per kilogram of the subject being treated. Yet another more preferred effective amount is between about 25 and about 50 micrograms per kilogram of the subject being treated. A most preferred effective amount is about 50 micrograms per kilogram of the subject being treated.

It was expected that effective amounts of wild type APC and APC-2Cys would be the same or similar, since they both have in common cytoprotective activity. It is also expected that effective amounts of other variants of APC with cytoprotective properties will be the same or similar to those observed for wild type APC or APC-2Cys, since the functional region or activity of the polypeptide responsible for the anti-adhesive activity is not altered. The skilled practitioner may also monitor post-dialysis events in the same or similar subjects and adjust the effective amount in subsequent surgeries. Examples of post-dialysis events that may be monitored include levels of the inflammatory indicators as described herein, namely TGF-β and tPA. Other examples of, inflammatory indicators include IL-1, IL-6, and TNFα. Inflammatory indicators are preferably monitored in the appropriate peritoneal fluid. Other post-dialysis indicators of fibrosis that may be measured include expression levels of cytokeratins, integrin2, matrix metalloproteinase 2 (MMP-2), and matrix metalloproteinase 9 MMP-9, as demonstrated in the examples. Post-dialysis events that may be monitored also include physiological symptoms of fibrosis associated with peritoneal dialysis. By way of example, reduced efficiency of the dialysis treatment in that particular subject. A skilled practitioner may increase the amount of APC administered subsequently, if the previous amounts are insufficient, or if a further reduction in fibrosis desired.

An appropriate volume of aqueous solution is that which would be treat the peritoneal cavity of a particular subject. This volume is not expected to be critical and may be approximated by the treating physician. By way of example it is expected that 100 ml may be an appropriate volume of an adult human. By way of another example, APC (wild type) or a variant of APC with cytoprotective activity, by way of example APC 2Cys, may be added directly to the dialysate if treatment concurrent with dialysis is desired.

### III. Subjects

Subjects, as used herein, are meant to include human and non-human mammals. It is expected that human subjects will benefit greatly from the invention. It is also expected that non-human mammalian subjects, including experimental animals, will benefits for the further development of therapies using peritoneal dialysis.

The discussion of the references herein is intended merely to summarize the assertions made by the authors and no admission is made that any reference constitutes prior art. Applicants reserve the right to challenge the accuracy and pertinence of the cited references.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

### Methods and Materials

Mouse Model of Peritoneal Fibrosis

The Inventors used 20 eight-week old male C57bl/6 mice (Jackson Labs), divided into two groups, using 10 mice for control group and 10 mice for APC treatment group. An injection of 0.1% of the drug chlorhexidine gluconate (CG) (known to induce peritoneal fibrosis), dissolved in 0.2 ml of saline containing 15% ethanol, was given intraperitoneally every day for 21 days. One hour before administrating chlorhexidine gluconate each day, 50 μg/kg of recombinant APC was injected intraperitoneally in 0.2 ml of an aqueous solution. At 10 and 21 days after the injections commenced, the mice were sacrificed and the parietal peritoneum and omentom were carefully dissected for the histological and expression of inflammatory molecules evaluation.

### Histological Evaluation

Following sacrificing, mice parietal peritoneum, and omentum were separated from other tissues, fixed in 10% formalin, and immersed in paraffin. Several paraffin sections were made by microtome. These sections were stained using Haematoxylin-Eosin (H&E) and Masson Trichrome. The areas of peritoneal fibrosis were assessed in predetermined fields (×200) of the mesothelial compact zone captured by a digital camera and the area stained was determined using a Lumina Vision 2.20 (Olympus, USA) in 10 fields. The number of available cells accumulated in the submesothelial compact zone was counted in 10 fields.

### RNA Extraction and Quantitative

Real-Time Reverse Transcription-peR

Total RNA was extracted using Aurum™ Total RNA Mini Kit (Bio-Rad, Hercules, Calif.) and 1 pg of the total RNA was reverse-transcribed with the high-capacity cDNA reverse transcription kit (Applied Biosystems, Foster City, Calif.) according to the manufacturer's instructions. The reactions were incubated in a thermal cycler for 10 min at 25° C., 120 min at 37° C., 5 min at 85° C. and then held at 4° C. Real-time quantitative PCR (qPCR) was run on a LightCycler Roche 480 with the SYBR Advantage qPCR Premix (Clontech, Mountain View, Calif.). The amplification was performed with an initial denaturation at 95° C. for 30 s, followed by 40 cycles of denaturation at 95° C. for 5 s and combined annealing/extension at 63° C. for 30 s. A melt-analysis was run for all products to evaluate the specificity of the amplification. Results were normalized to the expression level of GAPDH and presented as the fold difference relative to the control group at each time point using the 2-6Ct method. Primer sets for individual genes were designed using the Primer 3 program from the Whitehead Institute for Biomedical Research/MIT Center for Genome Research at hltp://www'.genome.wi.mit.edu and synthesized by IDT (Integrated DNA Technologies, Coralville, Iowa).

Enzyme-Linked Immunosorbent Assay

Commercially available ELISA kits were used to measure the peritoneal fluid concentrations of cytokines, tissue plasminogen activator (tP A) and tissue growth factor beta-I (TGF-β1) (eBiosciense, USA).

Statistical Analysis

Results are expressed as the mean±SD. Statistical analysis was performed using Student's test and P<0.05 was considered to be statistically significant.

### Example 1

The results of hematoxylin-eosin staining presented in FIG. 1 demonstrate that APC (wild type APC) will inhibit peritoneal fibrosis. Mice injected with only CG exhibit a dramatic increase in the thickening of the peritoneum by day 21 (FIG. 2, microphotographs A and B). By contrast, in the APC-treated group, the CG-induced thickening of the peritoneum was completely inhibited by day 21 (FIG. 1 microphotographs B, C). Quantitative analysis of cell density and peritoneum thickening, (FIG. 2, bar graphs A, B) further illustrate that APC is a potent inhibitor of CG-induced peritoneal fibrosis. Studies using Masson's trichrome staining indicated that the thickened peritoneum present in the CG-control (CG only) group contains a significant amount of collagen (FIG. 3B) by day 21. In the CG-APC treatment group, APC (wild type) completely inhibited the expression of the collagen by peritoneal cells (FIG. 3C) during the same time period (21 days).

### Example 2

It is known that enhanced expression of tumor growth factor beta (TGF-β) plays an important role in causing excessive collagen synthesis in the extracellular matrix and tissue fibrosis, thereby leading to formation of a pathological fibrosis in tissues lining the peritoneal cavity. In support of APC inhibiting CG or dialysis-induced peritoneal fibrosis through the inhibition of TGF-β signaling pathway, it was demonstrated that APC (wild type) inhibited the expression of TGF-β mRNA in CG-treated mice (FIG. 4A). Interestingly, further studies revealed that APC also inhibits the mRNA expression levels of cytokeratins (FIG. 4B), integrin2 (FIG. 4C), matrix metalloproteinase 2 (MMP-2) (FIG. 4D) and MMP-9 (FIG. 4E) in the CG-treated mice. Consistent with the anti-inflammatory role of APC through down-regulation of the metalloproteinase proteolytic pathway in this model, APC (wild type) markedly enhanced the mRNA expression level of tissue inhibitor of metalloproteinase 2 (TIMP2) (FIG. 4F). Furthermore, APC (wild type) mediated the enhanced expression of the protein level of anti-fibrosis protease tissue-type plasminogen activator (tPA) following both day 10 (FIG. 5A) and day 21 (FIG. 5B) in CG-treated mice. Similarly, APC (wild type) inhibited the protein level of TGF-β in CG-treated mice in both day 10 (FIG. 5C) and day 21 (FIG. 5D) of the experiments. Taken together, these results clearly demonstrate that APC potently inhibits peritoneal fibrosis in this mouse model.

Examples 1 and 2 using wild type APC demonstrate that an intraperitoneal administration of APC will inhibit a fibrosis mediated thickening of the peritoneum as well as a fibrosis mediated inflammatory processes.

### Example 3

The Inventors believed that an APC polypeptide with cytoprotective activity but lacking anti-coagulant activity would reduce the incidence of peritoneal fibrosis. In support of this hypothesis the Inventors tested a variant of APC, which possessed these properties. The variant, designated APC-2Cys was tested under the same conditions described for wild type APC. Similar to the results obtained for wild type APC in Example 1, hematoxylin-eosin staining revealed that mice, injected with only CG, exhibited a dramatic increase in the thickening of the peritoneum by day 21 (FIG. 6, panel A), whereas, in the APC-2Cys treated group, CG-induced thickening of the peritoneum was dramatically inhibited by day 21 (FIG. 6 panel A). Quantitative analysis of these results further illustrates that APC-2Cys is a potent inhibitor of peritoneal fibrosis (FIG. 6 panels B and C).

### Example 4

In further support for APC polypeptides with cytoprotective activity but lacking anti-coagulant activity to reduce the peritoneal fibrosis, the Inventors examined tissue-type plasminogen activator (tPA) and transforming growth factor beta (TGF-□□ levels in CG treated mice. Similar to Example 2 using wild type APC, it was shown that APC-2Cys treatment increased levels tPA at day-10 (FIG. 7, panel A) and day-21 (FIG. 7, panel B), and decreased levels of TGF-□ on both day-10 (FIG. 7, panel C) and day-21 (FIG. 7, panel D). In CG-treated mice. Both an increase in tPA and a decrease in TGF-β are consistent with a reduction peritoneal fibrosis.

The results of Examples 5 and 6 indicate that APC-2Cys is equally effective as wild type APC in reducing the incidence of fibrosis. Also demonstrated is that anticoagulant activity is not required since APC-2Cys lacks this function. Common to both APC polypeptides are cytoprotective or anti-inflammatory activity.

All publications and patents cited in this specification are hereby incorporated by reference in their entirety. The discussion of the references herein is intended merely to summarize the assertions made by the authors and no admission is made that any reference constitutes prior art. Applicants reserve the right to challenge the accuracy and pertinence of the cited references.

What is claimed is:

1. A method of reducing the incidence of peritoneal fibrosis, associated with peritoneal dialysis, in a subject, the method comprising, injecting the subject intraperitoneally with an effective amount of an activated protein C having cytoprotective or anti-inflammatory activity, within a time, reasonably close to when peritoneal dialysis is to be administered.

2. The method of claim 1, wherein the activated protein C possess reduced anticoagulant activity.

3. The method of claim 1, wherein the effective amount is about 25 micrograms per kilogram to about 200 micrograms per kilogram of the subject being treated.

4. The method of claim 1, wherein the effective amount is about 25 micrograms per kilogram to about 50 micrograms per kilogram of the subject being treated.

5. The method of claim 1, wherein the effective amount is about 50 micrograms per kilogram of the subject being treated.

6. The method of claim 1, wherein the time, reasonably close to when peritoneal dialysis is administered is the same day as peritoneal dialysis is administered.

7. The method of claim 1, wherein the time, reasonably close to when peritoneal dialysis is administered is 4 hours or less, before or after peritoneal dialysis is administered.

8. The method of claim 1, wherein the time, reasonably close to when peritoneal dialysis is administered is concurrent with peritoneal dialysis, or 1 hour or less before or after peritoneal dialysis is administered.

9. The method of claim 1, wherein the subject exhibits reduced symptoms of fibrosis, associated with peritoneal dialysis.

10. The method of claim 1, wherein the subject exhibits improvement in the form of changes in levels of indicators for peritoneal fibrosis as measured in the peritoneal cavity fluid or peritoneal tissues.

11. The method of claim 10, wherein improvement in the form of a change in an indicator for peritoneal fibrosis is a decrease in the level of an indicator selected from the group consisting of TGF-β, cytokratins, integrin2, MMP2, and MMP9.

12. The method of claim 10, wherein improvement in the form of a change in an indicator for peritoneal fibrosis is an increase in the level of an indicator selected from the group consisting of tPA and TIMP2.

13. The method of claim 1 wherein activated protein C consist of Drotrecogin Alfa Activated.

14. The method of claim 1 wherein the activated protein C consist of SEQ ID NO:1, containing conservative amino acid substitutes, secreted from an eukaryotic cell, and activated in vitro, wherein the activated protein C possesses cytoprotective or anti-inflammatory activity.

15. The method of claim 1 wherein activated protein C consist of SEQ ID NO:1 secreted from an eukaryotic cell, and activated in vitro.

16. The method of claim 1 wherein the activated protein C consist of SEQ ID NO:2, containing conservative amino acid substitutes, secreted from an eukaryotic cell, and activated in vitro, wherein the activated protein C possesses cytoprotective or anti-inflammatory activity.

17. The method of claim 1 wherein activated protein C consist of SEQ ID NO:2 secreted from an eukaryotic cell, and activated in vitro.

18. The method of claim 1 wherein the subject is a human subject.

19. A method of reducing the incidence of peritoneal fibrosis, associated with peritoneal dialysis, in a human subject undergoing peritoneal dialysis, the method comprising, injecting the subject intraperitoneally with an effective amount of SEQ ID NO:1 secreted from an eukaryotic cell, and activated in vitro, 1 hour before the time peritoneal dialysis is to be administered.

20. A method of reducing the incidence of peritoneal fibrosis associated with peritoneal dialysis, in a human subject undergoing peritoneal dialysis, the method comprising, injecting the subject intraperitoneally with an effective amount of SEQ ID NO:2 secreted from an eukaryotic cell, and activated in vitro, 1 hour before the time peritoneal dialysis is to be administered.


SEQUENCE LISTING (8 sequences)

SEQ ID NO: 1 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 2 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVCLGEYDLRRWEKWELCLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 3 - DNA, 33 bp, Homo sapiens
aagaagctccttgtctgccttggagagtatgac

SEQ ID NO: 4 - DNA, 33 bp, Homo sapiens
gtcatactctccaaggcagacaaggagcttctt

SEQ ID NO: 5 - DNA, 33 bp, Homo sapiens
gagaagtgggagctgtgcctggacatcaaggag

SEQ ID NO: 6 - DNA, 33 bp, Homo sapiens
ctccttgatgtccaggcacagctcccacttctc

SEQ ID NO: 7 - DNA, 1776 bp, Homo sapiens
gctgtcatggcggcaggacggcgaacttgcagtatctccacgacccgcccctacaggtgccagtgcctccagaatgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggatgggacattaaagggacatgtaacaagcacaccggcctgctgttctgtccttccatccctcttttgggctcttctggagggaagtaacatttactgagcacctgttgtatgtcacatgccttatgaatagaatcttaactcctagagcaactctgtggggtggggaggagcagatccaagttttgcggggtctaaagctgtgtgtgttgagggggatactctgtttatgaaaaagaataaaaaacacaaccacgaaaaaaaaaaaaaaaaaaaaaaa

SEQ ID NO: 8 - DNA, 1776 bp, Homo sapiens
gctgtcatggcggcaggacggcgaacttgcagtatctccacgacccgcccctacaggtgccagtgcctccagaatgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtctgccttggagagtatgacctgcggcgctgggagaagtgggagctgtgcctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggatgggacattaaagggacatgtaacaagcacaccggcctgctgttctgtccttccatccctcttttgggctcttctggagggaagtaacatttactgagcacctgttgtatgtcacatgccttatgaatagaatcttaactcctagagcaactctgtggggtggggaggagcagatccaagttttgcggggtctaaagctgtgtgtgttgagggggatactctgtttatgaaaaagaataaaaaacacaaccacgaaaaaaaaaaaaaaaaaaaaaaa